(19) United States
(12) Patent Application Publication
(10) Pub. No.: US 2009/0200399 A1
(43) Pub. Date: Aug. 13, 2009

(54) Volatile Liquid Dispensing Device

(75) Inventors: Thomas McGee, Cottesloe (AU); Colin Litten-Brown, Bracknell Berkshire (GB); Kishen Gohil, Surrey (GB); Guy Edward Naish, Bicester Oxfordshire (GB)

Correspondence Address: NORRIS, MCLAUGHLIN & MARCUS, 875 THIRD AVE, 18TH FLOOR, NEW YORK, NY, 10022, US

(73) Assignee: Givaudan SA, Vernier (CH)

(21) Appl. No.: 12/299,521

(22) PCT Filed: Apr. 27, 2007
(86) PCT No.: PCT/CH2007/000206

§ 371 Date: Nov. 21, 2008

Related U.S. Application Data
(60) Provisional application No. 60/798,235, filed on May 5, 2006.

(51) Int. Cl. A61L 9/03; B05B 1/24

(52) U.S. Cl. 239/136; 239/282; 239/302; 392/390

(57) ABSTRACT

A plug-in device adapted to disseminate a plurality of volatile liquids, such as fragrances, individually into an atmosphere, the device comprising a plurality of volatile liquids and a dissemination means, the liquids being accommodated on a rotatable wheel, which wheel is rotated such that the individual liquids come into liquid disseminating proximity to the liquid disseminating means. The device substantially overcomes the problem of “habituation” encountered with a single fragrance air freshener.

This invention relates to a device for dispensing multiple fragrances.

Devices for disseminating into an atmosphere an active volatile liquid are well known to the art. By “active” is meant a liquid whose presence in the atmosphere is desired for the performance of some function—typical examples include a fragrance, a disinfectant, an odour masking agent or a fungicide. A wide variety of such devices is commercially available. While most of these rely on evaporation alone, many have assisted dissemination, in that the evaporation is augmented by a heating element, a forced air current or both. These are generally powered by electricity, supplied by internal or external batteries, solar cells or mains power. One common and particularly desirable type of augmented evaporation device is the so-called “plug-in” type, a relatively small device that comprises, in a single unit, reservoir, dissemination element (such as a porous wick), electrically-driven augmentation element (such as heater and/or fan), transformer and power outlet pins, allowing it to be plugged directly into a power outlet without requiring a power lead. These are cheap and popular. However, most of these disseminate only one liquid.

It is advantageous to be able to disseminate two or more different liquids sequentially. For example, in the field of air fresheners, it is known that people become accustomed to a single fragrance (the art describes this as “habituated”) and it loses its effectiveness. Dissemination of a second, different fragrance after a suitable time can counter this. It may also be useful to disseminate entirely different liquids, for example, a fungicide, followed by a fragrance to mask the odour of the fungicide.

It has now been found that it is possible by using a simple device. The invention therefore provides a plug-in device adapted to disseminate a plurality of volatile liquids individually into an atmosphere, the device comprising a plurality of volatile liquids and a liquid dissemination means, the liquids being accommodated on a rotatable wheel, the device being configured such that rotation of the wheel brings the liquids individually into liquid disseminating proximity to the liquid disseminating means.

By “dissemination means” is meant some element that assists in the dissemination of the liquid, that is, dissemination is not solely reliant on unassisted evaporation. The two most common such means are forced air circulation by means of a fan or blower, and a heating element. Both of these means may be used in a single apparatus.

The device is a plug-in device, that is, it is designed to be plugged directly into a power point without the need for a power cord. The device thus comprises the necessary plug pins and, where necessary, a transformer to convert the mains voltage to one suitable for powering the heating element or the source of forced air current, and if desired, the rotatable wheel. Heating elements are well known to the art and are readily available. The types already in use on such apparatus as air fresheners are generally suitable. Any such heating element should be located in liquid-emitting proximity to the wheel, that is, it is placed close enough to the wheel that it will cause the dissemination of volatile liquid from a cavity near thereto. With regard to forced air current, the small blowers used in laptop computers are suitable.

The plurality of volatile liquids is accommodated on a rotatable wheel. Volatile liquids accommodated on rotating cartridges are known to the art, for example, those described in US published applications 2002/0068010 and 2004/0009103. While undoubtedly effective, such devices are large and mechanically complex, and they are never plug-in devices. The cartridges used therein are also complex and relatively expensive items.

The rotatable wheel of the present invention is circular and essentially flat and comprises a plurality of cavities arranged around the wheel at a uniform distance from the centre of the circle. Each cavity holds a volatile liquid rendered unable to flow out of the cavities by any suitable means, for example, by covering the cavity openings with a suitable semi-permeable membrane or by incorporating the liquids in a gel or in a sublimable substance. The wheel may be cheaply made of known raw materials by known means. Typical materials include metals, ceramics, cellulosic materials and plastics. The nature of the wheel may vary with varying forms of the device, but in its simplest form it is a solid disc of material in which cavities have been created by any convenient means, such as stamping, moulding, drilling, routing and cutting. The cavities may be created in the disc itself, or they may be added, for example, by making perforations in the wheel and inserting containers of liquids into the perforations. New wheels may be made available as refills, so that the device can be easily replenished when the liquids are exhausted. In addition, different wheels with different liquids may also be provided, such that all personal preferences may be accommodated.

The wheel is rotatably mounted in the device and may be rotated by hand. Hand rotation may be arranged by any convenient means. For example, the wheel may be arranged such that an edge thereof is able to be touched and moved, but there are many other ways of achieving this. Alternatively, it may be rotated by an electric motor that draws power from the power point that powers the dissemination means. The motor may act either directly or indirectly, for example, via gearing, drive band or contacting rubber-faced wheel acting against another wheel or the wheel rim itself. It is possible to provide several indirect means, such as several sets of gears, such that the speed of rotation can be changed. The speed of the motor itself can also be varied electronically. It is possible to provide for both electrical and manual rotation, such that, for example, the electrical rotation can be overridden, so that individual choice of liquid to be disseminated may be accommodated.

The device is configured with respect to physical dimensions and speed of rotation such that liquid is disseminated essentially from only one cavity at a time. Minor dissemination from cavities adjacent to the liquid-disseminating is tolerable. As is the case with the heating element, the air current should be configured such that it impinges essentially on one cavity at a time. However, again, some minor overlap is tolerable. In the case of the heating element embodiment, one way of ensuring the heating of only one cavity at a time is to provide a wheel of non-heat-conductive material with perforations, giving it essentially the appearance of a spoked wheel. Into the perforations are fitted metal trays containing the liquids. The heating element is of such a size relative to the portions of the wheel separating the cavities (the “spokes” of the wheel) that, at some part of the revolution of the wheel, the heating element is not in liquid-emitting contact with any cavity but only with the wheel. This substantially prevents the dissemination of two liquids simultaneously.

In both cases, the wheel may be suitably marked, for example, on a visible edge, so that the particular liquid being disseminated can be readily identified (and if desired changed).

In some embodiments, the device may be configured such that the order in which, and the time over which, individual liquids are disseminated may be predetermined. One way is, as outlined above, to provide the motor turning the wheel with several different reduction gearings, such that a suitable rotation speed may be selected. Another possibility is a heating element or a supply of air current that is regulable, such that at least two different temperatures and/or air velocities are possible. In another embodiment, there is used, instead of or in addition to the reduction gearing, programmable electronics. Suitable chips are readily available and can be easily incorporated into the device. Programming may be done by means of a keypad on the device itself or by a remote control, either connected to the device or wireless. Such means confers on a small, cheap device a hitherto unattainable versatility—liquids can be selected according to desire and personal preferences can be easily accommodated. While it is simpler to disseminate liquids in the order in which simple rotation brings them into proximity with the dissemination means, the particular order of dissemination brought about thereby may not always be desirable, and the programmable device allows this order to be changed at will.

The device of the present invention has many advantages. It offers most of the versatility of the commercially-available sophisticated devices in a small, convenient and cheap apparatus. The wheels are easily and inexpensively made and can be easily replaced.

The invention is now further described with reference to the drawings, which depict particular embodiments of the invention and which are not intended to be in any way limiting on the scope of the invention. The device depicted is an air freshener adapted to disseminate fragrance into an ambient atmosphere.

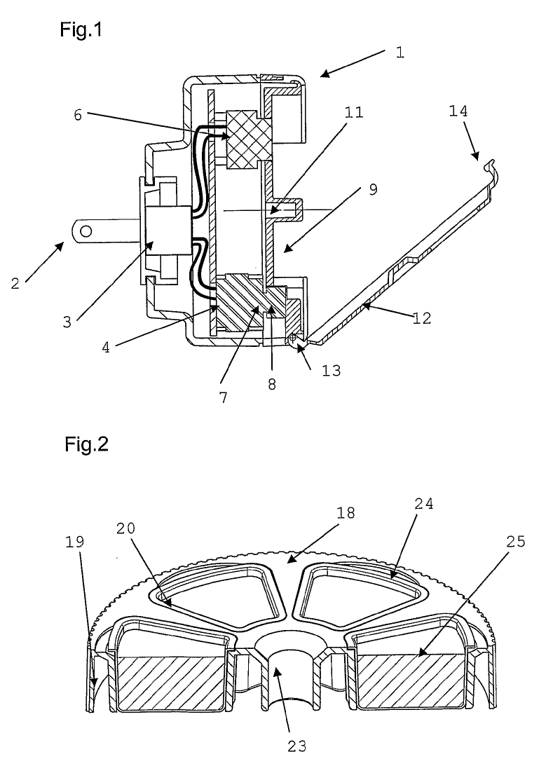

FIG. 1 is a schematic front-to-back cross-section of an embodiment of the invention without fragrances.

FIG. 2 is a perspective cross-section of a fragrance-containing wheel adapted to be used in a device of FIG. 1.

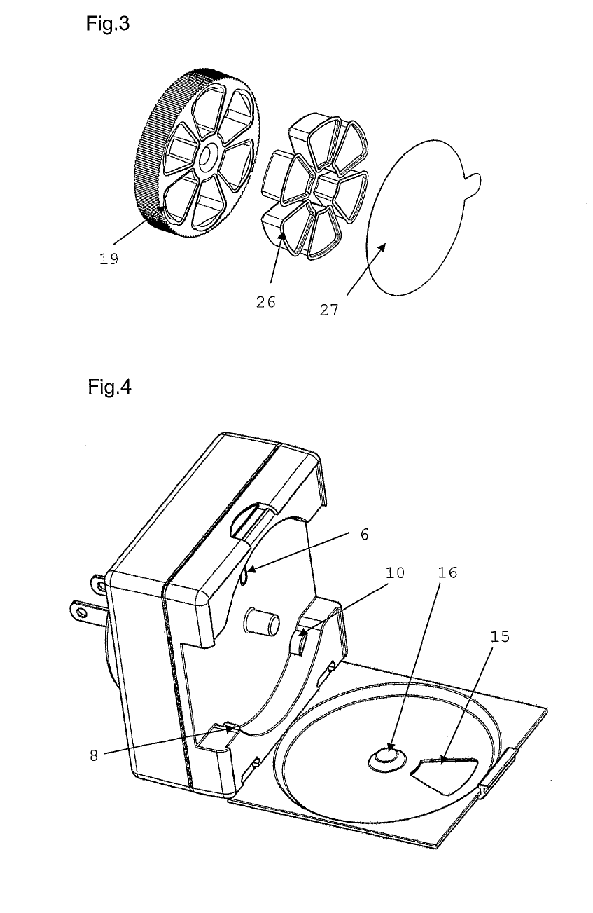

FIG. 3 is an exploded perspective view of a fragrance wheel according to FIG. 2.

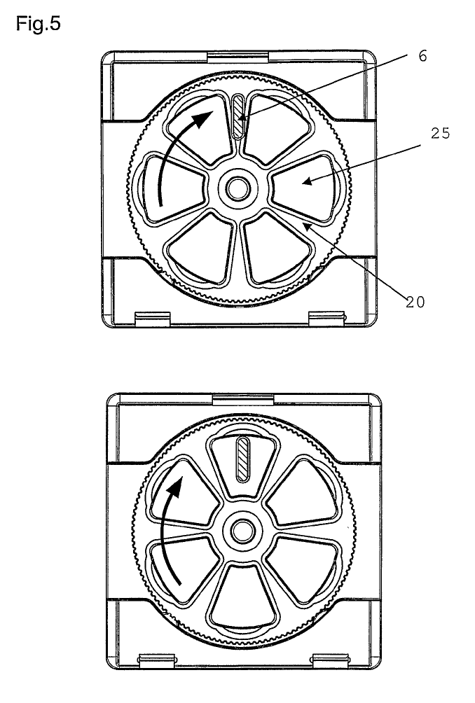

FIG. 4 is a perspective view of the device of FIG. 1.

FIG. 5 is a schematic view of a fragrance wheel mounted in an apparatus according to FIG. 1.

In FIGS. 1 and 4, a device has a body 1 essentially in the form of a box. It is equipped at the back with electricity pins 2, adapted to be fitted into a power point. The pins convey electricity to a transformer 3 and thence to an electric motor 4 and a heating element 6. The motor drives a gearbox (not shown), which in turn drives a rubber-faced cylindrical roller 8 via a shaft 7. The roller is located in the device such that it contacts the circumferential edge of a circular wheel 18 that is rotatably mounted on a horizontal shaft 11 in a cavity 9 of matching shape in the device. The wheel 18 is mounted such that the rotating roller 8 causes it to rotate. The cavity also comprises a further roller 10 that also contacts the edge of the wheel, this being a free-wheeling one to assist in the proper rotation of the wheel. The heating element 6 has an exposed surface in the cavity, this being positioned to promote the dissemination of volatile liquid, as will hereinafter be described.

To secure the wheel in place, the cavity comprises a lid 12, attached by a hinge 13 along a front bottom edge of the body 1, such that it hinges upwards to cover the open end of the cavity 9. To keep the wheel in place, the lid is equipped with a small wheel 16 mounted on a helical spring (not shown) and arranged to press against the wheel when the lid is closed. When closed, it is held thus by a releasable catch 14. The lid additionally comprises a port 15, facing the heating element 6 when it is closed and leading from the interior of the cavity to the atmosphere, this allowing the volatile liquid to escape.

The wheel 18 comprises cavities 19 formed therein. These are distributed uniformly around the centre of the wheel, such that, in this particular case, the wheel has the form of a spoked wheel with spokes 20 separating the cavities 19. The spokes are dimensioned so that they are the same width as the surface of the heating element 6 in the cavity and as the port 15 in the lid 12. A hole 23 in the centre of the wheel allows mounting of the wheel on the shaft 11. (This hole may also include a bearing). Between each pair of spokes is fitted a metal tray 24 containing a volatile liquid 25. In the embodiment of FIG. 3, the trays are formed in a single unit 26, able to be fitted into a wheel. The individual liquids are kept in the trays by semi-permeable membranes (not shown) and premature evaporation is prevented by a peelable protective film 27.

In operation, the peelable film 27 is removed, the wheel is mounted on the shaft 11 and the lid 12 is closed. When the device is plugged into an electricity supply, the heating element 6 commences to heat and the wheel starts to rotate. As each cavity comes into alignment with the heating element, the heat causes volatile liquid to evaporate and pass through the semi-permeable membrane and the port 15 to the atmosphere. This happens to each liquid in sequence. The fact that the spokes 20 and the port 15 are the same width as the heating element 6 in the cavity ensures that the simultaneous evaporation of two different liquids is effectively zero. FIG. 5 illustrates this.

The skilled person will be able to envisage many other embodiments of this invention all of which fall within the scope of this invention.

1. A plug-in device adapted to disseminate a plurality of volatile liquids individually into an atmosphere, the device comprising a plurality of volatile liquids and a liquid dissemination means, the liquids being accommodated on a rotatable wheel, the device being configured such that rotation of the wheel brings the liquids individually into liquid disseminating proximity to the liquid disseminating means.

2. A device according to claim 1, in which the wheel is manually rotatable.

3. A device according to claim 1, in which the wheel electrically rotatable.

4. A device according to claim 1, in which the disseminating means is a heating element.

5. A device according to claim 4, in which the wheel is of non-heat-conducting material and has essentially the form of a spoked wheel, the spokes being wider than the heating element, the liquids being contained in trays fitting into the gaps between the spokes.

6. A device according to claim 5, in which the trays are of metal and the wheel of a non-heat-conductive material.
